(19) United States
(12) Patent Application Publication
(10) Pub. No.: US 2007/0184093 A1
(43) Pub. Date: Aug. 9, 2007

(54) Dissolving films

(76) Inventor: Jung Hang, Winnipeg (CA)

Correspondence Address: Ade & Company;PO Box 28006, 1795 Henderson Highway, Winnipeg, MB, R2G OP1, CA

(21) Appl. No.: 10/564,679

(22) PCT Filed: Dec. 22, 2003
(86) PCT No.: PCT/CA03/01958

§ 371 Date: Oct. 24, 2006

Related U.S. Application Data
(60) Provisional application No. 60/436,013, filed on Dec. 26, 2002.

(51) Int. Cl. A61K 9/70

(52) U.S. Cl. 424/443

(57) ABSTRACT

The preparation of soluble films composed of a first soluble polymer and a second strengthening polymer is described.

The present application claims priority on U.S. Ser. No. 60/435,013, filed Dec. 26, 2002.

### FIELD OF THE INVENTION

The present invention relates generally to the field of dissolving films.

### BACKGROUND OF THE INVENTION

A variety of water-soluble or dissolving films are disclosed in the prior art. For example:

U.S. Pat. No. 6,419,903 teaches a rapidly dissolvable orally consumable film for delivering breath freshening agents to the oral cavity. The film is formed from a mixture of water soluble, low viscosity hydroxyalkylmethyl cellulose, a water dispersible starch and a flavoring agent. The hydroxyalkylmethyl cellulose serves as a strong film former to give the film the necessary mechanical strength and maintain the integrity of the film at elevated temperatures. The starch ingredient increases the stiffness of the film and reduces curling of the film. The hydroxyalkyl cellulose to starch ratio (by weight) varies from about 1:3 to about 4:1.

PCT Application WO 00/18365 teaches edible films comprising pullulan and a number of essential oils.

U.S. Pat. No. 5,047,244 teaches an anhydrous but hydratable polymer matrix and amorphous fumed silica combination which may further include a water-insoluble film which is applied thereon for providing a non-adhering surface.

U.S. Pat. No. 6,284,264 teaches a mucoadhesive film comprised of a hydrophillic and/or water dispersible polymer or mixtures thereof. U.S. Pat. No. 6,177,096 teaches a film having instant wettability followed by rapid dissolution/disintegration upon administration in the oral cavity.

### SUMMARY OF THE INVENTION

According to a first aspect of the invention, there is provided a soluble film comprising:

a soluble polymer, selected from the group consisting of microcrystalline cellulose, (pre-)gelatinized starch, modified starch, dextrin, maltodextrin, pectin, iota-carrageenan, lambda-carrageenan, gum arabic, gum acacia, gum ghatti, guar gum, xanthan gum, gellan gum, pullulan and combinations thereof; and

a mechanically strong polymer selected from the group consisting of modified cellulose (carboxymethyl-, methyl-, hydroxypropyl-, hydroxypropylmethyl-), (pre-)gelatinized high amylose starch, agar, alginates, kappa-carrageenan, furcellaran, gum karaya, gum tragacanth, locust bean gum, chitosan and mixtures thereof.

According to a second aspect of the invention, there is provided a combination comprising:

a pouch composed of:

a soluble polymer, selected from the group consisting of microcrystalline cellulose, (pre-)gelatinized starch, modified starch, dextrin, maltodextrin, pectin, iota-carrageenan, lambda-carrageenan, gum arabic, gum acacia, gum ghatti, guar gum, xanthan gum, gellan gum, pullulan and combinations thereof; and

a mechanically strong polymer selected from the group consisting of modified cellulose (carboxymethyl-, methyl-, hydroxypropyl-, hydroxypropylmethyl-), (pre-)gelatinized high amylose starch, agar, alginates, kappa-carrageenan, furcellaran, gum karaya, gum tragacanth, locust bean gum, chitosan and mixtures thereof; and

a unit dosage within the pouch.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Described herein is a dissolvable film comprising a first polymer and a second polymer. The film has a variety of uses, for example, but by no means limited to, as an orally dissolvable strip or as a dissolvable pouch, as described below or as dissolvable films or sheets arranged to dissolve on contact with aqueous environments.

Depending on the intended use, the formulation may include other components, for example, but by no means limited to, pharmaceutical agents, antimicrobial agents, nutraceutical ingredients, plasticizers, surfactants, colorants, sweetening agents, flavors, flavor enhancers and other excipients.

In one embodiment, the film is used for the delivery of a wide range of pharmaceutically active ingredients. These include for example but are by no means limited to sedatives, nutraceuticals, antiepileptics, psychoneurotropic agents, neuromuscular blocking agents, antispasmodic agents, antihistaminics, antiallergics, antiarrhythmics, diuretics, hypotensives, vasopressors, thyroid hormones, antidiabetics, antitumor agents, antibiotics, chemotherapeutics and narcotics. For example, The fast dissolving film will be a suitable delivery system for emergency medical care situations such as strong pain killer delivery or nitroglycerin delivery. As will be appreciated by one of skill in the art, these pharmaceutically active agents may be present within the film at a therapeutically effective amount, that is, at an amount sufficient to have the desired effect, which may vary according to for example patient age, weight and condition.

Suitable plasticizers include but are by no means limited to monosaccharides, disaccharides, sugar alcohols and polyols, for example, glucose, fructose, glycerol, sorbitol, polyethylene glycol, propylene glycol, natural honey and corn syrup and mixtures thereof. As will be appreciated by one of skill in the art, plasticizers are any ingredients which reduce the glass transition temperature of polymers which in turn increase flexibility, solubility, water absorptiveness and wettability of the end product. For example, the addition of plasticizers in dissolving films enhances the water-related properties of the films, which include hygroscopicity, hydrophilicity, solubility, degradation. in aqueous systems and flexibility.

Suitable surfactants include any food grade surfactants, for example, but by no means limited to fatty acid monoglycerides, span, tween, polysorbates and lecithin.

Suitable sweeteners include sweeteners that have low solubility and high tendency to form sugar crystals. As a consequence, these sweeteners are recommended to be used at below 10% (w/w dry solid). Examples of such sweeteners include but are by no means limited to: sucrose, mannitol, galactose, xylose, lactose and mixtures thereof.

As will be appreciated by one of skill in the arts, other sweeteners having higher solubilities may be added at any suitable concentration according to the intended use of the end product. Suitable sweeteners include but are by no means limited to glycerol, sorbitol, fructose, glucose, maltose, maltitol, mannose and mixtures thereof. It is of note that these sugars act also as plasticizers, as discussed above.

As will be appreciated by one of skill in the art, an excess of plasticizers will make the end film product rubbery or a sticky gel and will also interfere with the drying process. Given that sweeteners (and also any other additives) can function as plasticizers, in some embodiments, the amount of plasticizers is controlled according to the total amount of sugars. The composition of plasticizers, sugars (sweeteners) and polymers varies with respect to the plasticizing effectiveness of additives and film forming ability of polymers. However, in most cases, the 1:1 (w/w) of polymer: other ingredients including sweeteners, plasticizers and other bioactive ingredients, is preferred, but the invention is not limited to this ratio. For optimal mechanical properties of dissolving films, this 1:1 ratio may be modified.

In other embodiments, artificial sweeteners are used in preparation of the dissolving films, since they possess stronger sweetness compared to the natural sweeteners, for example, at a concentration of 0.1%-10% or under the gelation concentration. As a result, a lower amount of the artificial sweeteners may be used which avoids the crystallization problems present with some of the sweeteners, as discussed above. It is of note that any commercial artificial sweetener for example, microcrystalline cellulose, carboxy methyl cellulose, or (pre-)gelatinized starch, is suitable for use with the dissolving films.

Examples of suitable flavours include but are by no means limited to oils of lavender, basil, oregano, rose flower, rosemary, garlic, ginger, cinnamon, horseradish, mustard and wasabi; and water extracts of ginger, garlic, cinnamon, green tea, ginseng, coffee, and citrus (grapefruit, orange, mandarin orange, lemon, lime). It is of note that these flavours are for illustrative purposes only and any suitable food flavour may be used in preparation of the dissolving films. As will be appreciated by one of skill in the art, the loss of volatile flavors during drying can be limited by using delivery agents such as for example α-, β-, γ-cyclodextrin, maltodextrin, modified starches, various benzoic acids and food grade emulsifiers.

In other embodiments, various benzoic acids (including propyl parabenzoic acid), potassium sorbate, sorbic acid, calcium sulfate, lactic acid, acetic acid, citric acid, lysozyme, nisin, cetyl pyrimidium chloride and tri-sodium phosphate may be mixed with the film materials as antimicrobials agents. The mixing of these agents with the film forming solutions caused no problems and exhibited antimicrobial activities against common pathogens. It is of note that other food grade anti-microbial additives, for example, preservatives known in the art may be added to the dissolving films.

Delivering agents can be mixed with the film forming solutions as well as with the volatile active agents such as flavors, pharmaceuticals, nutraceuticals, antimicrobials, prior to the active agents being mixed with the film forming solutions.

The first polymer is a very soluble film forming polymer or gelating polymer. In some embodiments, the first polymer is present at a concentration of 0.1% to 5% or in some embodiments, 0.5% to 5% or in other embodiments, 1% to 5%. In yet other embodiments, the upper concentration corresponds to the gelation concentration of the final film-forming solution. As will be apparent to one of skill in the art, the gelation concentration is the concentration at which a given polymer forms a strong gel, and is dependent upon the characteristics and purity of the given polymer. Examples of suitable polymers include for example but are by no means limited to microcrystalline cellulose, (pre-)gelatinized starch, modified starch, dextrin, maltodextrin, pectin, iota-carrageenan, lambda-carrageenan, gum arabic, gum acacia, gum ghatti, guar gum, xanthan gum, gellan gum, pullulan and combinations thereof.

As will be appreciated by one of skill in the art, gelation concentration of the above polymers varies with the purity of the polymer raw materials. Below the gelation concentration, film forming solutions form a very viscous liquid and are easily applied on any flat surface or food surface. However, above the gelation concentration, the film forming solution becomes a soft, solid gel. Heating is then required to form a flow of the soft solid gel in order to spray the gelated film forming solution onto a flat surface. Preferred polymers to produce a dissolving film are carboxymethyl cellulose, modified starches, dextrin, pectin, gellan gum, pullulan, since they can produce very soluble films without adding the mechanically strong second polymers.

The second polymer is a mechanically strong film forming polymer or insoluble film forming polymer. In some embodiments, the second polymer is added at a concentration of 0% to 10%, in other embodiments, 0.1% to 10%, in yet other embodiments, 0.5% to 10% or in yet other embodiments, 1% to 10%. It is of note that in yet other embodiments the upper concentration range of the second polymer is under the gelation concentration of the final film forming solution. Suitable second polymers include for example but are by no means limited to modified cellulose (methyl-, hydroxypropyl-, hydroxypropylmethyl-), (pre-)gelatinized high amylose starch, agar, alginates, kappa-carrageenan, furcellaran, gum karaya, gum tragacanth, locust bean gum, chitosan and mixtures thereof.

As will be apparent to one of skill in the art, the second polymers produce very strong films. These films are hydrophilic but less soluble in water compared to the first polymers. Thus, the greater the proportion of the second polymer in the mixture of polymers, the lower the solubility of the final film product.

The ratio of the 1^{st} soluble polymer and the 2^{nd} mechanically strong polymer may be varied for producing end products having varied properties. For example, increasing the amount of the 1st soluble polymer will increase the water solubility and total soluble matter of the end product but will decreases strength and stiffness of the final product. On the other hand, increasing the amount of the 2nd mechanically strong polymer will decrease the water solubility and total soluble matter of the end product, but will increase strength and stiffness. For example, the additon of kappa carrageenan (2nd polymer) into carboxymethyl cellulose, modified starches, dextrin, pectin, gellan gum or pullulan increase the film strength but decrease the solubility and total soluble matter. The dissolution rate also decreases. This formulation will be suitable for slower release modification but may leave slimy insoluble matter. However, the addition of maltodextrin or iota/lambda carrageen into the same above polymers will increase the solubility and total soluble matter. Thus, increasing the amount of the soluble 1^{st} polymer will increase the dissolution rate of the end product, but will decrease the film strength. Increasing the amount of the 1^{st} polymer past a certain limit may result in the film structure being disintegrated at high moisture conditions. Thus, varying the quantity and/or ratio of the 1st and 2nd polymers permits the modification of the film structure. It is of note that in some instances, the 1^{st} polymer may in fact be a mixture of two or more soluble polymers as discussed above and/or the 2^{nd} polymer may in fact be a mixture of two or more mechanically strong polymers as discussed above.

It is of note that because most polymers are negatively charged or neutral, the use of positively charged polymers (such as chitosan and positively charged modified starch) with negatively charged polymers is not recommended as this would create severe coagulation problem between polymers, and result in water-insoluble precipitation. The positively charged polymers can however be mixed with neutral polymers. Accordingly, there is the proviso that negatively charged polymers are not mixed with positively charged polymers.

As an example, Maltodextrin cannot form a film. Instead, it forms a viscous solution. After drying, it turns to powder. Similarly, honey can not create a film and it takes a very long time to dry honey. Once honey is dried, it turns to solid. When these materials are mixed with high amylase starch or kappa carrageenan, these first polymers (maltodextrin, honey) increase the water solubility of the second polymers (high amylase starch, kappa carrageenan) resulting in dissolving films. High amylase starch and kappa carrageenan produce swell-able films with low solubility without addition of the first polymers.

Carboxymethyl cellulose, gelatinized starch, pectin, gellan gum and pullulan produce a very dissolving film. When the second polymers are mixed with suitable first polymers, the final films possess less solubility in water and stronger mechanical strength than those films containing the first polymer alone.

Therefore, the ratio of the first polymer and the second polymer should be optimized based on the desired mechanical properties and water solubility of the final product. That is, the optimum ratio of 1st polymer and 2nd polymer for a given end product depends on the final application of the end product. For example, high solubility and fast dissolution are required for fast delivery application and/or fast dissolving applications, while low solubility is required for slow release and longer dissolving time applications. For example, 100% caboxymethyl cellulose, pullulan, pectin or gellan gum produce fairly soluble films. But 50%:50% of the above film forming a polymer with hydroxypropylmethyl cellulose produces swell-able films, which is not completely soluble in water. The film would be very strong in stretching and puncturing. On the other hand, 50%:50% of the above film forming soluble polymer with iota (or lambda) carrageenan produces a very slimy and sticky film which dissolves in water very fast and does not leave any insoluble-matter.

As discussed above, edible films are known in the art (see for example, U.S. Pat. No. 6,177,096, U.S. Pat. No. 6,419,903, U.S. Pat. No. 6,284,264 and PCT WO00/18365 which are incorporated herein by reference). As will be appreciated by one of skill in the art, a film that is too moist is difficult to handle or process and may also take considerable time to dry. In addition, if the film is mechanically weak, it is difficult to handle and/or process. Furthermore, such a film, if processed, often feels undesirably slimy when taken orally. The instant invention overcomes this by combining two polymers, a first soluble polymer and a second strengthening polymer. As discussed above, the ratio of the polymers is critical so that an acceptable is produced, as discussed above.

For use, the additional ingredients are mixed into water, with the exception that if any ingredients are not soluble in water, the insoluble ingredients are first dissolved in ethanol or edible oil separately, and then mixed with the ingredient solution. As will be apparent to one of skill in the art, in embodiments where insoluble ingredients are used, the use of surfactant(s) to form a homogeneous emulsion, colloid or suspension is very important.

Typically, the first polymer and the second polymer are dissolved in water separately. However, if there is no strong interaction or coagulation between the first polymer and the second polymer, both polymers can be dissolved in water together. It is of note that warm or hot water can be used for easy dissolution, and that heating of the polymer solution helps the dissolution process and polymer gelatinization.

In most instances, the first polymer solution is mixed into the active ingredient solution first, and then the second polymer solution is added. In some case, if there is not significant polymer-polymer interaction or coagulation, the two polymer solutions can be mixed with active ingredient solution at the same time, as discussed above.

The film forming solution (whether water-based, ethanol based or colloid solution) is spread on non-sticky smooth surface and dried to form a film. The non-sticky surface materials are, for example, but by no means limited to, PE, Teflon, polished hard rubber, greased metal, greased glass, or a plastic (PE or Teflon) coated metal. Two types of drying processes can be applied, which are (1) a batch process using plates, and (2) a continuous process using conveyor belts. The non-sticky materials described above may be used for the main materials of the plates or the conveyor belts. As will be appreciated by one of skill in the art, the drying condition will vary based on the thermal sensitivity and evaporation of active ingredients.

In some embodiments, a soluble or dissolvable pouch is prepared instead of a film. In these embodiments, the film forming solution is prepared as described above. The film forming solution is then casted on a flat, non-sticky smooth surface and dried to form a film. The film can be used with regular pouch making machines known in the art. It is of note that most of the films prepared as described above are thermoplastic, meaning that heat sealing can be used to form a pouch.

Alternatively, the film polymers or combinations may be fed into film making extruders to form molten films, which are commercially used for plastic film production. Compared to wet casing methods described above using film-forming solution, the extrusion process minimizes the use of solvents such as water, ethanol or edible oils. Therefore, the concentration of the ingredients should be adjusted according to the process used. However, changes to the ratio of 1st polymer and 2nd polymer are not necessary, as the same polymer ratio which has been used above can still be utilized. The only change in the formulation is the amount of solvent. Solvent content should be reduced and the total solid content increased accordingly. For the extrusion process, it is recommended that the water content in the film forming material (polymers+plasticizers+other additves+water) be 10% -50%.

As will be apparent to one of skill in the art, the soluble pouch can be used advantageously for storing unit doses of a variety of products, for example, medicines, nutraceuticals or food ingredients. The pouches would be ideally suited for the food industry, fast-food restaurants and the military as the soluble pouches are arranged to contain premeasured quantities of ingredients, food, and, in the case of hospitals, medicines. As will be apparent to one of skill in the art, in this context, “unit dose” or “unit dosage” refers to a quantity sufficient for a single use. The actual size of the unit dose may vary considerably based upon the intended use.

The film product should be stored in low humidity packages made by relatively high moisture barriers such as polyolefin plastic containers. Since the films are totally soluble in water, they will be dissolved instantly in human mouth or any water existing condition.

The first polymers are rapidly soluble compared to the second polymer. Among the first polymers, those which are mostly soluble in any pH, negatively charged polymers are more soluble in neutral & alkali pH than acid pH such pH sensitive polymers are modified starch & pectin.

It is of note that all of the first and second polymers are edible and biodegradable, and soluble in the most common conditions of human consumptions such as body temperature, saliva, blood, liquid foods, and medicine.

When polymer concentrations over the gelation concentration are used (as well as over 5%), the syrupy, highly viscous or gelated polymers can be sprayed under high pressure conditions. The gelated polymers can be fluidized by increased temperature and high pressure. The polymers can be applied through a nozzle and the water can be removed easily by vaporization during the spray process. Recommended pressures are 0 psi˜50 psi. As discussed above, the same ratio between 1st polymer and 2nd polymer which has been used for the wet-casting method can be used in the spray applications. Since this spray method uses high pressure, a slight reduction in the amount of water in the film forming material does not create any problem for the process. If the pressure can create liquid flow of the film forming material, it is acceptable to decrease the water content.

While sprayers work with any concentration, at concentrations above gelation, only a high pressure sprayer is suitable. Below the gelation concentration, dipping, brushing and any other transferring apparatus is suitable. If the polymer concentration increases, the sprayer requires higher pressure to make a flow of gel with heating. Therefore, for these applications, the maximum concentration is the concentration which the sprayer can make a flow of the gel with the given sprayer pressure.

Emulsifiers and surfactants were mixed with the film forming solutions, but they can also be sprayed on the film surface after the film forming solution is applied on the drying plates. They can be sprayed on the film surface any time during or after the drying process. The main purpose of spraying is to prevent the film surface from being sticky due to moisture absorption during the manufacturing process and distribution.

The spray is for embodiments containing at least one emulsifier and at least one surfactant. Emulsifiers & surfactant can be mixed with other ingredients or applied during film drying process by a sprayer separately. There could be two methods for emulsifiers & surfactants. One is mixing these with other film forming ingredients, and the other is spraying these during or after drying process. The second methods, therefore, should be equipped by the secondary sprayer for the surface spray of emulsifiers & surfactants. This should be a fine mist sprayer to minimize the thickness of emulsifiers & surfactants on the surface of dried films.

The invention will now be described by way of examples. However, the examples are for illustrative purposes only.

### EXAMPLE 1

### Soluble Oral Strip Containing Vitamin C

Solution 1—vitamin C solution (10% ascorbic acid in water), 100 ml cold water;

Solution 2—1% carboxy-methyl-cellulose (CMC) and 0.5% glycerol, complete dissolution in 100 ml hot water

Solution 2 is heated with stirring to completely dissolve the CMC. After dissolution, CMC/glycerol solution is cooled down to room temperature.

Solution 1 and solution 2 are mixed with slow agitation.

The mixed film-forming solution is poured onto a flat plastic plate (PE, teflon or hard rubber), a greased glass plate, a greased metal plate, a plastic (PE or Teflon) coated metal plate, or a flat conveyor belt made by any of the above materials (PE, Teflon, hard rubber, greased metal, plastic (PE or Teflon) coated metal.

The plates are placed in a drying oven at 50-60° C. After complete drying, the film is peeled off from the plate.

### EXAMPLE 2

### Soluble Pouch for Baking Process

Solution: 1% gellan gum+1% pre-gelatinized high amylase starch+1% plasticizer in 100 mL warm water.

If the high amylose starch is not pre gelatinized, the above dispersion should be boiled to gelatinize starch. Glycerol, sorbitol or glucose can be used as a plasticizer. The above solution will be transferred on drying plates or conveyer made by the materials described in example 1, then placed in a drying oven at 100° C.

After complete drying the film will be fold and sealed to form a opened pouch. This pouch now can contain premeasured yeast, baking powers or other baking ingredients, and final-sealed to close the pouch intact. At the baking factory, this pouch can be mixed with other ingredients without measurement of weight since it contains pre-measured ingredients. The above formula can also contain various emulsifiers and surfactants.

### EXAMPLE 3

### Cinnamon Flavored Oral Sanitizing Strip

Solution 1: Cinnamon extract <10%+Emulsifier (polysorbate or span) 0.5% (optional)+cetyl pyrimidium chloride <1%+artificial sweetener (aspartame, saccharin or others) <0.1% in 100 mL warm water

Solution 2: 1%-5% pectin in 100 mL warm or hot water+glycerol (20% to 50% of pectin)

Process: After formulating solution 1 and solution 2, these two solutions are mixed together with gentle agitation. Solution 1 and solution 2 could be prepared together in a same container; however, it may take a longer time to form a homogeneous film forming solution than separating preparation. Drying process of example 1 should follow after the film forming solution is prepared. Since cinnamon extract is less volatile extract, the drying temperature can be increased to 80-90° C. Instead of cetyl pyrimidium chloride, various phenolic compounds can be used to obtain sanitizing function.

### EXAMPLE 4

### General Formula

The first polymer: 1˜5%
The second polymer: 0.1˜5%
Plasticizer: 50˜150% of polymers
Emulsifier: 0˜10% of polymers or 0˜10% of any Oil-soluble ingredients
Active ingredients and delivery agents: 0%-98%, or the maximum concentration dispersible
Water: q.v.a. 100%
Total: 100%

The above formula is a generally suggested formula. This formula can be modified to optimize the mechanical strength and water-solubility of final films.

In the majority of the examples discussed above, the dissolving strip or film has a thickness above 0.1 mm. The thickness is controllable by altering the polymer concentration or by changing the amount of film forming solution on the unit area of drying plate.

While the preferred embodiments of the invention have been described above, it will be recognized and understood that various modifications may be made therein, and the appended claims are intended to cover all such modifications which may fall within the spirit and scope of the invention.

1. A soluble film comprising:
a soluble polymer, selected from the group consisting of microcrystalline cellulose, (pre-)gelatinized starch, modified starch, dextrin, maltodextrin, pectin, iota-carrageenan, lambda-carrageenan, gum arabic, gum acacia, gum ghatti, guar gum, xanthan gum, gellan gum, pullulan and combinations thereof; and
a mechanically strong polymer selected from the group consisting of modified cellulose (carboxymethyl-, methyl-, hydroxypropyl-, hydroxypropylmethyl-), (pre-)gelatinized high amylose starch, agar, alginates, kappa-carrageenan, furcellaran, gum karaya, gum tragacanth, locust bean gum, chitosan and mixtures thereof.

2. The soluble film according to claim 1 wherein the soluble polymer is present at a concentration below the gelation concentration of said soluble polymer.

3. The soluble film according to claim 1 wherein the mechanically strong polymer is present at a concentration below the gelation concentration of said mechanically strong polymer.

4. The soluble film according to claim 1 including a therapeutically active agent.

5. The soluble film according to claim 4 wherein the therapeutically active agent is a pharmaceutical compound.

6. The soluble film according to claim 4 wherein the therapeutically active agent is a nutraceutical compound.

7. The soluble film according to claim 1 including an antimicrobial agent.

8. The film according to claim 1 in a pouch form.

9. A combination comprising:
a pouch composed of:
a soluble polymer, selected from the group consisting of microcrystalline cellulose, (pre-)gelatinized starch, modified starch, dextrin, maltodextrin, pectin, iota-carrageenan, lambda-carrageenan, gum arabic, gum acacia, gum ghatti, guar gum, xanthan gum, gellan gum, pullulan and combinations thereof; and
a mechanically strong polymer selected from the group consisting of modified cellulose (carboxymethyl-, methyl-, hydroxypropyl-, hydroxypropylmethyl-), (pre-)gelatinized high amylose starch, agar, alginates, kappa-carrageenan, furcellaran, gum karaya, gum tragacanth, locust bean gum, chitosan and mixtures thereof; and
a unit dosage within the pouch.

10. The combination according to claim 9 wherein the unit dosage is of a pharmaceutical compound.

11. The combination according to claim 9 wherein the unit dosage is of a food product.

12. The combination according to claim 9 wherein the unit dosage is of a food industry ingredient.

13. The soluble film according to claim 9 wherein the soluble polymer is present at a concentration below the gelation concentration of said soluble polymer.

14. The soluble film according to claim 9 wherein the mechanically strong polymer is present at a concentration below the gelation concentration of said mechanically strong polymer.

15. The soluble film according to claim 9 including a therapeutically active agent.

16. The soluble film according to claim 15 wherein the therapeutically active agent is a pharmaceutical compound.

17. The soluble film according to claim 15 wherein the therapeutically active agent is a nutraceutical compound.

18. The soluble film according to claim 9 including an antimicrobial agent.
